(19) United States
(12) Patent Application Publication
(10) Pub. No.: US 2004/0219165 A1
(43) Pub. Date: Nov. 4, 2004

(54) Streptococcus pneumoniae antigens

(75) Inventors: Allan William Cripps, Nicholls (AU); Jennelle Maree Kyd, McKellar (AU); Maha Jomaa, Theodore (AU); Jeremy Mark Wells, McKellar (AU); Phillip Michael Hansbro, Newcastle (AU)

Correspondence Address: STERNE, KESSLER, GOLDSTEIN & FOX PLLC, 1100 NEW YORK AVENUE, N.W., WASHINGTON, DC, 20005, US

(73) Assignee: Provalis UK Limited, Flintshire (GB)

(21) Appl. No.: 10/859,548

(22) Filed: Jun. 3, 2004

Related U.S. Application Data
(63) Continuation of application No. 09/962,863, filed on Sep. 26, 2001, now abandoned.
Application No. 09/962,863 is a continuation of application No. PCT/GB00/01167, filed on Mar. 27, 2000.

(30) Foreign Application Priority Data
Mar. 26, 1999 (GB) 9907114.4
Dec. 3, 1999 (GB) 9928678.3

(51) Int. Cl. A61K 39/02; C07K 14/315

(52) U.S. Cl. 424/190.1; 530/350

(57) ABSTRACT

There are provided various novel antigens from Streptococcus pneumoniae, as well as homolgoues, derivatives and fragments thereof. The use of these in medicine is described, particularly in the treatment or prophylaxis of S. pneumoniae infections. The use of the antigens in diagnosis is also described.

The present invention relates to proteins derived from Streptococcus pneumoniae, nucleic acid molecules encoding such proteins, the use of the nucleic acid and/or proteins as antigens/immunogens and in detection/diagnosis, as well as methods for screening the proteins/nucleic acid sequences as potential anti-microbial targets.

Respiratory diseases remain a major cause of morbidity and mortality throughout the world. Streptococcus pneumoniae is a major causative pathogen in the respiratory tract. Infections caused by this pathogen include otitis media, lower respiratory tract infections, bacteremia and meningitis.

Streptococcus pneumoniae, commonly referred to as the pneumococcus, is an important pathogenic organism. The continuing significance of Streptoccocus pneumoniae infections in relation to human disease in developing and developed countries has been authoritatively reviewed (Fiber, G. R., Science, 265: 1385-1387 (1994)). That indicates that on a global scale this organism is believed to be the most common bacterial cause of acute respiratory infections, and is estimated to result in 1 million childhood deaths each year, mostly in developing countries (Stansfield, S. K, Pediatr. Infect. Dis., 6: 622 (1987)). In the USA it has been suggested (Breiman et al, Arch. Intern. Med., 150: 1401 (1990)) that the pneumococcus is still the most common cause of bacterial pneumonia, and that disease rates are particularly high in young children, in the elderly, and in patients with predisposing conditions such as asplenia, heart, lung and kidney disease, diabetes, alcoholism, or with immunosupressive disorders, especially AIDS. These groups are at higher risk of pneumococcal septicaemia and hence meningitis and therefore have a greater risk of dying from pneumococcal infection. The pneumococcus is also the leading cause of otitis media and sinusitis, which remain prevalent infections in children in developed countries, and which incur substantial costs.

The need for effective preventative strategies against pneumococcal infection is highlighted by the recent emergence of penicillin-resistant pneumococci. It has been reported that 6.6% of pneumoccal isolates in 13 US hospitals in 12 states were found to be resistant to penicillin and some isolates were also resistant to other antibiotics including third generation cyclosporins (Schappert S. M., Vital and Health Statistics of the Centres for Disease Control/National Centre for Health Statistics, 214:1 (1992)). The rates of penicillin resistance can be higher (up to 20%) in some hospitals (Breiman et al, J. Am. Med. Assoc., 271: 1831 (1994)). Since the development of penicillin resistance among pneumococci is both recent and sudden, coming after decades during which penicillin remained an effective treatment, these findings are regarded as alarming.

The burden of disease caused by these pathogens is highly significant and contributes significantly to national health budgets. Although there is a vaccine available for Streptococcus pneumoniae, this vaccine is not highly efficacious in children under two years. Current therapy relies on antibiotic treatment of the infection. Many suffering from infections caused by Streptococcus pneumoniae live in developing countries, where some communities have very limited access to adequate medical treatment. Thus, antibiotic treatment may not be available. In the developed world, where antibiotics are available, there has been a significant emergence of antibiotic resistance in these bacteria.

The development of an effective vaccine against S. pneumoniae is therefore a desirable objective. In particular, it is desirable to develop a vaccine which can be used in young children.

Various approaches have been taken in order to provide vaccines for the prevention of pneumococcal infections. Difficulties arise for instance in view of the variety of serotypes (at least 90) based on the structure of the polysaccharide capsule surrounding the organism. Vaccines against individual serotypes are not effective against other serotypes and this means that vaccines must include polysaccharide antigens from a whole range of serotypes in order to be effective in a majority of cases. An additional problem arises because it has been found that the capsular polysaccharides (each of which determines the serotype and is the major protective antigen) when purified and used as a vaccine do not reliably induce protective antibody responses in children under two years of age, the age group which suffers the highest incidence of invasive pneumococcal infection and meningitis.

A modification of the approach using capsule antigens relies on conjugating the polysaccharide to a protein-in order to derive an enhanced immune response, particularly by giving the response T-cell dependent character. This approach has been used in the development of a vaccine against Haemophilus influenzae, for instance. There are, however, issues of cost concerning both the multi-polysaccharide vaccines and those based on conjugates.

A third approach is to look for other antigenic components which offer the potential to be vaccine candidates. This is the basis of the present invention. We have now identified a number of proteins from S.pneumoniae which are antigenic/immunogenic.

Thus, in a first aspect the present invention provides a protein or polypeptide obtainable from S.pneumoniae selected from:

(i) one having a molecular weight of 55 kDa, as determined by SDS/PAGE, and having the N-terminal sequence VEPKAKPADPSVV;

(ii) one having a molecular weight of 50 kDa, as determined by SDS/PAGE, and having the N-terminal sequence NDRLVATQSADGRNESVLMSIET;

(iii) one having a molecular weight of 85 kDa, as determined by SDS/PAGE, and having the N-terminal sequence EDTTNSRFGSQFDKYRQPNAEPDHSHDAVSADNSTAHNRFGYGFAIGSKYIRY D;

(iv) one having a molecular weight of 38 kDa, as determined by SDS/PAGE, and having the N-terminal sequence DKYRQPNAEPDDHHYAV;

(v) one having a molecular weight of 30 kDa, as determined by SDS/PAGE, and having the-N-terminal sequence DAVSAD or SETNVY;

(vi) one having a molecular weight of 32 kDa, as determined by SDS/PAGE, and having the N-terminal sequence DKVDGLSAKPDILKP;

(vii) one having-a molecular weight of 43 kDa, as determined by SDS/PAGE, and having the N-terminal sequence ELKEEG(W)VVK;

(viii) one having a molecular weight of 100 kDa, as determined by SDS/PAGE, and having the N-terminal sequence EVHA;

(ix) one having a molecular weight of <14 kDa, as determined by SDS/PAGE, and having the N-terminal sequence MKLNEVKEFVKELRAET;

(x) one having a molecular weight of <14 kDa, as determined by SDS/PAGE, and having the N-terminal sequence AKYEILYIERPNIEEFAK;

(xi) one having a molecular weight of <14 kDa, as determined by SDS/PAGE, and having the N-terminal sequence I(R)LTRM(E)GGKKKP(K)FYY;

(xii) one having a molecular weight of 16 kDa, as determined by SDS/PAGE, and having the N-terminal sequence VMTDPIADXLXRI;

(xiii) one having a molecular weight of 27.5 kDa, as determined by SDS/PAGE, and having the N-terminal sequence (VA)(KE)LVFARHGE(LT)E(NK);

(xiv) one having a molecular weight of 44 kDa, as determined by SDS/PAGE, and having the N-terminal sequence IITDVYAREVLDSRGNPTL.

(xv) one having a molecular weight of 12-14 kDa as determined by SDS PAGE under reducing conditions and has the following amino terminal sequence:

A L N I E N I I A E I K I A S
Ala Leu Asn Ile Glu Asn Ile Ile Ala Glu Ile Lys Ile Ala Ser

(xvi) is a reduced toxicity variant or fragment of the protein defined in (xv) above;

(xvii) one having a molecular weight of about 16 kDa as determined by SDS PAGE under reducing conditions; or

(xviii) has a molecular weight of about 57 kDa as determined by SDS PAGE under reducing conditions and has the following amino terminal sequence:

R I I K F V Y A K
Arg Ile Ile Lys Phe Val Tyr Ala Lys.

A protein or polypeptide of the present invention may be provided in substantially pure form. For example, it may be provided in a form which is substantially free of other proteins. In relation to the above quoted molecular weights, the skilled person will appreciate that slightly different results can be obtained in different hands or even on different occaisons in the same hands, thus, the molecular weight figures quoted herein should be read as ±5% or even ±10%.

As discussed herein, the proteins and/or polypeptides of the invention are useful as antigenic material. Such material can be “antigenic” and/or “immunogenic”. Generally, “antigenic” is taken to mean that the protein or polypeptide is capable of being used to raise antibodies or indeed is capable of inducing an antibody response in a subject. “Immunogenic” is taken to mean that the protein or polypeptide is capable of eliciting a protective immune response in a subject Thus, in the latter case, the protein or polypeptide may be capable of not only generating an antibody response but, in addition, a non-antibody based immune response.

The skilled person will appreciate that homologues or derivatives of the proteins or polypeptides of the invention will also find use in the context of the present invention, ie as antigenic/immunogenic material. Thus, for instance proteins or polypeptides which include one or more additions, deletions, substitutions or the like are encompassed by the present invention. In addition, it may be possible to replace one amino acid with another of similar “type”. For instance replacing one hydrophobic amino acid with another. One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention.

In the case of homologues and derivatives, the degree of identity with a protein or polypeptide as described herein is less important than that the homologue or derivative should retain its antigenicity or immunogenicity to Streptoccocus pneumoniae. However, suitably, homologues or derivatives having at least 60% similarity (as discussed above) with the proteins or polypeptides described herein are provided. Preferably, homologues or derivatives having at least 70% similarity, more preferably at least 80% similarity are provided. Most preferably, homologues or derivatives having at least 90% or even 95% similarity are provided.

In an alternative approach, the homologues or derivatives could be fusion proteins, incorporating moieties which render purification easier, for example by effectively tagging the desired protein or polypeptide. It may be necessary to remove the “tag” or it may be the case that the fusion protein itself retains sufficient antigenicity to be useful.

In an additional aspect of the invention there are provided antigenic fragments of the proteins or polypeptides of the invention, or of homologues or derivatives thereof.

For fragments of the proteins or polypeptides described herein, or of homologues or derivatives thereof the situation is slightly different. It is well known that is possible to screen an antigenic protein or polypeptide to identify epitopic regions, ie those regions which are responsible for the protein or polypeptide's antigenicity or immunogenicity. Methods for carrying out such screening are well known in the art Thus, the fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a protein or polypeptide, homologue or derivative as described herein. The key issue, once again, is that the fragment retains the antigenic/immunogenic properties.

Thus, what is important for homologues, derivatives and fragments is that they possess at least a degree of the antigenicity/immunogenicity of the protein or polypeptide from which they are derived.

The proteins may be obtained by extraction from S. pneumoniae and, therefore, in a further aspect of the invention, there is provided a process for the preparation of an isolated and purified protein the process comprising the following steps:

(a) preparing cultures of S. pneumoniae, growing the cultures under appropriate conditions and harvesting them, followed by washing with centrifugation to yield a washed cell pellet;

(b) resuspending the washed cells in an appropriate buffer followed by disruption of the cells;

(c) centrifuging to remove cell debris and obtaining the supernatant containing soluble cell proteins;

(d) subjecting the solution obtained to anion exchange chromatography with a sodium chloride gradient elution, and pooling the fractions corresponding to each separate peak;

(e) suspending the protein fractions in a buffer comprising 0.5 M Tris HCl pH 6.8; 10%(v/v) glycerol; 10% (w/v) SDS; 0.05% (w/v) bromophenol blue; and 0.05% (v/v) β-mercaptoetanol; boiling the mixture and then purifying by SDS-PAGE using a 12% (w/v) acrylamide/BIS separating gel with a 4% (w/v) acrylamide/BIS stacking gel, run at 16 mA in the stacking gel and 24 mA in the resolving gel;

(f) selecting a fraction containing a protein having a molecular weight of 12-14 kDa, 16 kDa, 34 kDa or 57 kDa and isolating the protein from the selected fraction.

Alternatively, gene cloning techniques may be used to provide a protein of the invention in substantially pure form. These techniques are disclosed, for example, in J. Sambrook et al Molecular Cloning 2nd Edition, Cold Spring Harbor Laboratory Press (1989). Thus, the N-terminal sequences of the proteins disclosed herein can in turn be used as the basis for probes to isolate the genes coding for the individual proteins. Thus, in another aspect the present invention provides a nucleic acid molecule comprising or consisting of a sequence which is:

(i) a DNA sequence coding for a protein or polypeptide as described herein or their RNA equivalents;

(ii) a sequence which is complementary to any of the sequences of (i);

(iii) a sequence which has substantial identity with any of those of (i) and (ii);

(iv) a sequence which codes for a homologue, derivative or fragment of a protein as defined herein.

The nucleic acid molecules of the invention may include a plurality of such sequences, and/or fragments. The skilled person will appreciate that the present invention can include novel variants of those particular novel nucleic acid molecules which are exemplified herein. Such variants are encompassed by the present invention. These may occur in nature, for example because of strain variation. For example, additions, substitutions and/or deletions are included. In addition and particularly when utilising microbial expression systems, one may wish to engineer the nucleic acid sequence by making use of known preferred codon usage in the particular organism being used for expression. Thus, synthetic or non-naturally occurring variants are also included within the scope of the invention.

The term “RNA equivalent” when used above indicates that a given RNA molecule has a sequence which is complementary to that of a given DNA molecule (allowing for the fact that in RNA “U” replaces “T” in the genetic code).

When comparing nucleic acid sequences for the purposes of determining the degree of homology or identity one can use programs such as BESTFIT and GAP (both from the Wisconsin Genetics Computer Group (GCG) software package) BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention compare when discussing identity of nucleic acid sequences, the comparison is made by alignment of the sequences along their whole length.

Preferably, sequences which have substantial identity have at least 50% sequence identity, desirably at least 75% sequence identity and more desirably at least 90 or at least 95% sequence identity with said sequences. In some cases the sequence identity may be 99% or above.

Desirably, the term “substantial identity” indicates that said sequence has a greater degree of identity with any of the sequences described herein than with prior art nucleic acid sequences.

It should however be noted that where a nucleic acid sequence of the present invention codes for at least part of a novel gene product the present invention includes within its scope all possible sequence coding for the gene product or for a novel part thereof

The nucleic acid molecule may be in isolated or recombinant form. It may be incorporated into a vector and the vector may be incorporated into a host Such vectors and suitable hosts form yet further aspects of the present invention.

Therefore, for example, by using probes designed on the basis of the N-terminal amino acid sequences described herein, genes in Streptococcus pneumoniae can be identified. They can then be excised using restriction enzymes and cloned into a vector. The vector can be introduced into a suitable host for expression.

Nucleic acid molecules of the present invention may be obtained from S.pneumoniae by the use of appropriate probes complementary to part of the sequences of the nucleic acid molecules. Restriction enzymes or sonication techniques can be used to obtain appropriately sized fragments for probing.

Alternatively PCR techniques may be used to amplify a desired nucleic acid sequence. Thus the sequence data-provided herein can be used to design two primers for use in PCR so that a desired sequence, including whole genes or fragments thereof can be targeted and then amplified to a high degree. One primer will normally show a high degree of specificity for a first sequence located on one strand of a DNA molecule, and the other primer will normally show a high degree of specificity for a second sequence located on the complementary strand of the DNA sequence and being spaced from the complementary sequence to the first sequence.

Typically primers will be at least 15-25 nucleotides long.

As a further alternative chemical synthesis may be used. This may be automated Relatively short sequences may be chemically synthesised and ligated together to provide a longer sequence.

As discussed herein, the inventors have also discovered that the 12-14kDa protein is a toxin which, if modified to reduce its toxicity, is likely to provide a highly efficacious vaccine. Strategies for defining the toxic portion of the protein include the preparation of sequentially truncated fragments or mutants.

As discussed herein, the proteins of the present invention as well as fragments and homologues thereof find use as immunogens. Thus, in an additional aspect, the present invention provides the use of the proteins of the invention, their homologues and/or fragments thereof in medicine, particularly in the prophylaxis and/or treatment of S.pneumoniae infections.

In yet a further aspect the present invention provides an immunogenic/antigenic composition comprising one or more proteins or polypeptides as described herein, or homologues or derivatives thereof, and/or fragments of any of these. In preferred embodiments, the immunogenic/antigenic composition is a vaccine or is for use in a diagnostic assay.

The vaccine composition may also comprise an adjuvant. Examples of adjuvants well known in the art include inorganic gels such as aluminium hydroxide or water-in-oil emulsions such as incomplete Freund's adjuvant Other useful adjuvants will be well known to the skilled man.

The protein may be administered by a variety of routes including enteral, for example oral, nasal, buccal, topical or anal administration or parenteral administration, for example by the intravenous, subcutaneous, intramuscular or intrapentoneal routes.

The form taken by the composition and the excipients it contains will of course, depend upon the chosen route of administration. For example, oral formulations may be in the form of syrups, elixirs, tablets or capsules, which may be enterically coated to protect the protein from degradation in the stomach. Nasal or transdermal formulations will usually be sprays or patches respectively. Formulations for injection may be solutions or suspensions in distilled water or another pharmaceutically acceptable solvent or suspending agent.

The appropriate dosage of the protein of the present invention to be administered to a patient will be determined by a clinician. However, as a guide, a suitable dose may be from about 0.5 to 20 mg per kg of body weight. It is expected that in most cases, the dose will be from about 1 to 15 mg per kg of body weight and preferably from 1 to 10 mg per kg of body weight. For a man having a weight of about 70 kg, a typical dose would therefore be from about 70 to 700 mg.

It is also possible to utilise the nucleic acid sequences described herein in the preparation of so-called DNA vaccines. Thus, the invention also provides a vaccine composition comprising one or more nucleic acid sequences as defined herein. The use of such DNA vaccines is described in the art. See for instance, Donnelly et al, Ann. Rev. Immunol., 15:617-648 (1997).

As already discussed herein the proteins or polypeptides described herein, their homologues or derivatives, and/or fragments of any of these, can be used in methods of detecting/diagnosing S.pneumoniae. Such methods can be based on the detection of antibodies against such proteins which may be present in a subject. Therefore the present invention provides a method for the detection/diagnosis of S.pneumoniae which comprises the step of bringing into contact a sample to be tested with at least one protein, or homologue, derivative or fragment thereof, as described herein. Suitably, the sample is a biological sample, such as a tissue sample or a sample of blood or saliva obtained from a subject to be tested.

In an alternative approach, the proteins described herein, or homologues, derivatives and/or fragments thereof, can be used to raise antibodies, which in turn can be used to detect the antigens, and hence S.pneumoniae. Such antibodies form another aspect of the invention. Antibodies within the scope of the present invention may be monoclonal or polyclonal.

Polyclonal antibodies can be raised by stimulating their production in a suitable animal host (e.g. a mouse, rat, guinea pig, rabbit, sheep, goat or monkey) when a protein as described herein, or a homologue, derivative or fragment thereof, is injected into the animal. If desired, an adjuvant may be administered together with the protein. Well-known adjuvants include Freund's adjuvant (complete and incomplete) and aluminium hydroxide. The antibodies can then be purified by virtue of their binding to a protein as described herein.

Monoclonal antibodies can be produced from hybridomas. These can be formed by fusing myeloma cells and spleen cells which produce the desired antibody in order to form an immortal cell line. Thus the well-known Kohier & Milstein technique (Nature 256 (1975)) or subsequent variations upon this technique can be used.

Techniques for producing monoclonal and polyclonal antibodies that bind to a particular polypeptide/protein are now well developed in the art. They are discussed in standard immunology textbooks, for example in Roitt et al, Immunology second edition (1989), Churchill Livingstone, London.

In addition to whole antibodies, the present invention includes derivatives thereof which are capable of binding to proteins etc as described herein. Thus the present invention includes antibody fragments and synthetic constructs. Examples of antibody fragments and synthetic constructs are given by Dougall et al in Tibtech 12 372-379 (September 1994).

Antibody fragments include, for example, Fab, F(ab′)₂ and Fv fragments. Fab fragments (These are discussed in Roitt et al [supra]). Fv fragments can be modified to produce a synthetic construct known as a single chain Fv (scFv) molecule. This includes a peptide linker covalently joining Vₕ and Vₗ regions, which contributes to the stability of the molecule. Other synthetic constructs that can be used include CDR peptides. These are synthetic peptides comprising antigen-binding determinants. Peptide mimetics may also be used. These molecules are usually conformationally restricted organic rings that mimic the structure of a CDR loop and that include antigen-interactive side chains.

Synthetic constructs include chimaeric molecules. Thus, for example, humanised (or primatised) antibodies or derivatives thereof are within the scope of the present invention. An example of a humanised antibody is an antibody having human framework regions, but rodent hypervariable regions. Ways of producing chimaeric antibodies are discussed for example by Morrison et al in PNAS, 81, 6851-6855 (1984) and by Takeda et al in Nature. 314,-452454 (1985).

Synthetic constructs also include molecules comprising an additional moiety that provides the molecule with some desirable property in addition to antigen binding. For example the moiety may be a label (e.g. a fluorescent or radioactive label). Alternatively, it may be a pharmaceutically active agent.

Antibodies, or derivatives thereof find use in detection/diagnosis of S.pneunoniae. Thus, in another aspect the present invention provides a method for the detection/diagnosis of S.pneumoniae which comprises the step of bringing into contact a sample to be tested and antibodies capable of binding to one or more proteins or polypeptides as described herein, or to homologues, derivatives and/or fragments thereof

In addition, so-called “Affibodies” may be utilised. These are binding proteins selected from combinatorial libraries of an alpha-helical bacterial receptor domain (Nord et al, ) Thus, Small protein domains, capable of specific binding to different target proteins can be selected using combinatorial approaches.

It will also be clear that the nucleic acid sequences described herein may be used to detect/diagnose S.pneumoniae. Thus, in yet a further aspect, the present invention provides a method for the detection/diagnosis of S.pneumoniae which comprises the step of bringing into contact a sample to be tested with at least one nucleic acid sequence as described herein. Suitably, the sample is a biological sample, such as a tissue sample or a sample of blood or saliva obtained from a subject to be tested. Such samples may be pre-treated before being used in the methods of the invention. Thus, for example, a sample may be treated to extract DNA. Then, DNA probes based on the nucleic acid sequences described herein (ie usually fragments of such sequences) may be used to detect nucleic acid from S.pneumoniae.

In additional aspects, the present invention provides:

(a) a method of vaccinating a subject against S.pneumoniae which comprises the step of administering to a subject a protein or polypeptide of the invention, or a derivative, homologue or fragment thereof, or an immunogenic composition of the invention;

(b) a method of vaccinating a subject against S.pneumoniae which comprises the step of administering to a subject a nucleic acid molecule as defined herein;

(c) a method for the prophylaxis or treatment of S.pneumoniae infection which comprises the step of administering to a subject a protein or polypeptide of the invention, or a derivative, homologue or fragment thereof, or an immunogenic composition of the invention;

(d) a method for the prophylaxis or treatment of S.pneumoniae infection which comprises the step of administering to a subject a nucleic acid molecule as defined herein;

(e) a kit for use in detecting/diagnosing S.pneumoniae infection comprising one or more proteins or polypeptides of the invention, or homologues, derivatives or fragments thereof, or an antigenic composition of the invention; and

(f) a kit for use in detecting/diagnosing S.pneumoniae infection comprising one or more nucleic acid molecules as defined herein.

Given that we have identified a group of important proteins, such proteins are potential targets for anti-microbial therapy. It is necessary, however, to determine whether each individual protein is essential for the organism's viability. Thus, the present invention also provides a method of determining whether a protein or polypeptide as described herein represents a potential anti-microbial target which comprises inactivating said protein or polypeptide and determining whether S.pneumoniae is still viable, in vitro or in vivo.

A suitable method for inactivating the protein is to effect selected gene knockouts, ie prevent expression of the protein and determine whether this results in a lethal change. Suitable methods for cawing out such gene knockouts are described in Li et al, P.N.A.S., 94:13251-13256 (1997).

In a final aspect the present invention provides the use of an agent capable of antagonising, inhibiting or otherwise interfering with the function or expression of a protein or polypeptide of the invention in the manufacture of a medicament for use in the treatment or prophylaxis of S.pneumoniae infection.

The invention will now be described with reference to the following example, which should not in any way be construed as limiting the scope of the invention

The examples refer to the figure in which:

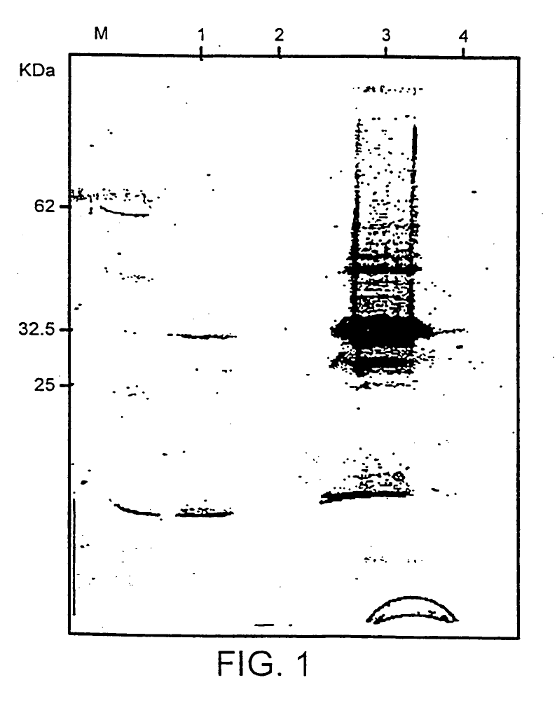

FIG. 1: shows a photograph of a 12% SDS PAGE gel of proteins extracted from cell wall material treated with 1 M solutions of 1. ammonium acetate, 2. ammonium chloride, 3. tri-methyl ammonium chloride or 4. Tris-HCl (pH 6.8).

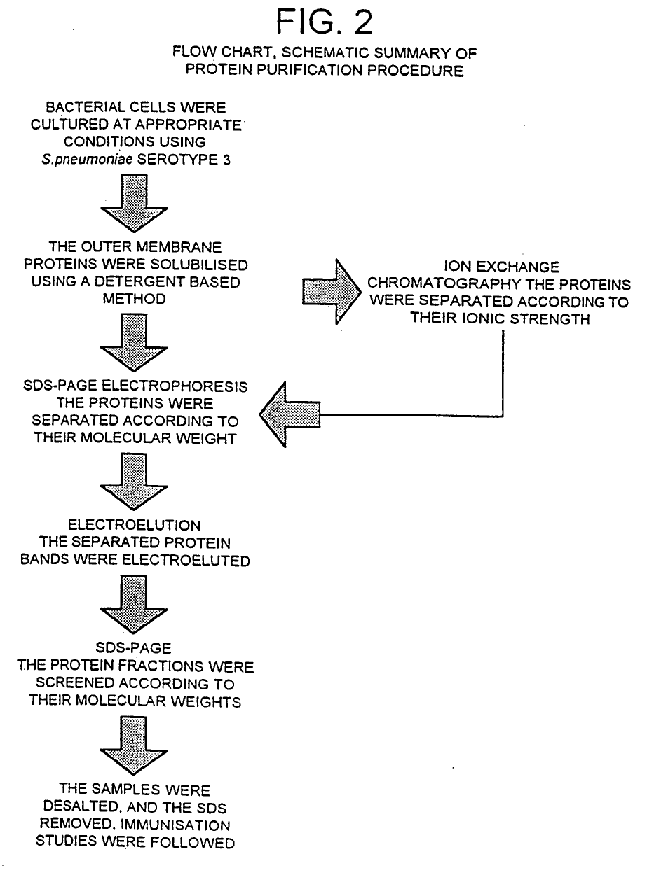

FIG. 2: is a flow chart with a schematic summary of the protein purification procedure used in the present invention.

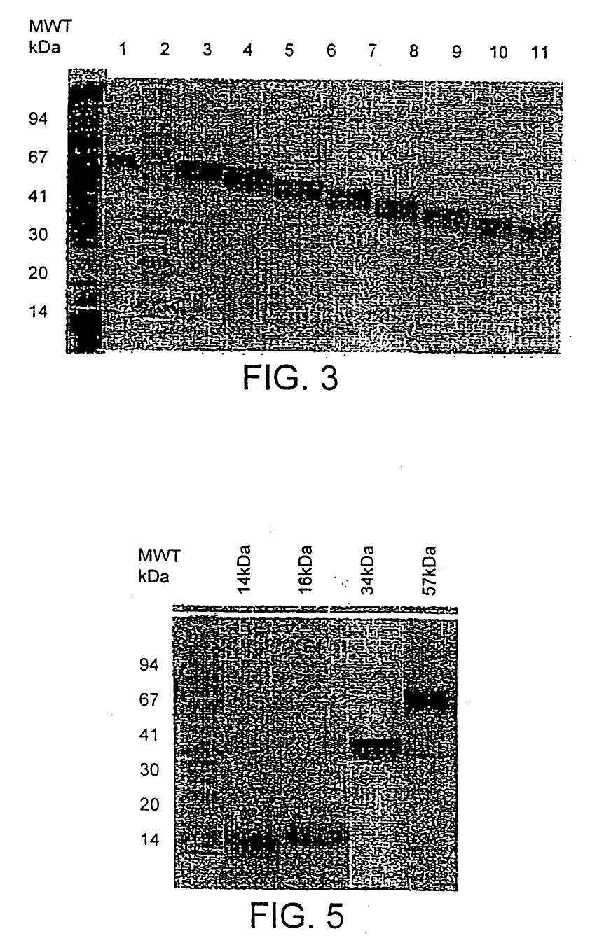

FIG. 3: is the electroelution profile from S. pneumoniae cell wall extract analysed on SDS-PAGE.

Lane 1: coomassie stain of crude extract separated by SDS-PAGE;

Lane 3: molecular mass standards;

Lanes 2 and 4-11: proteins recovered by electroelution.

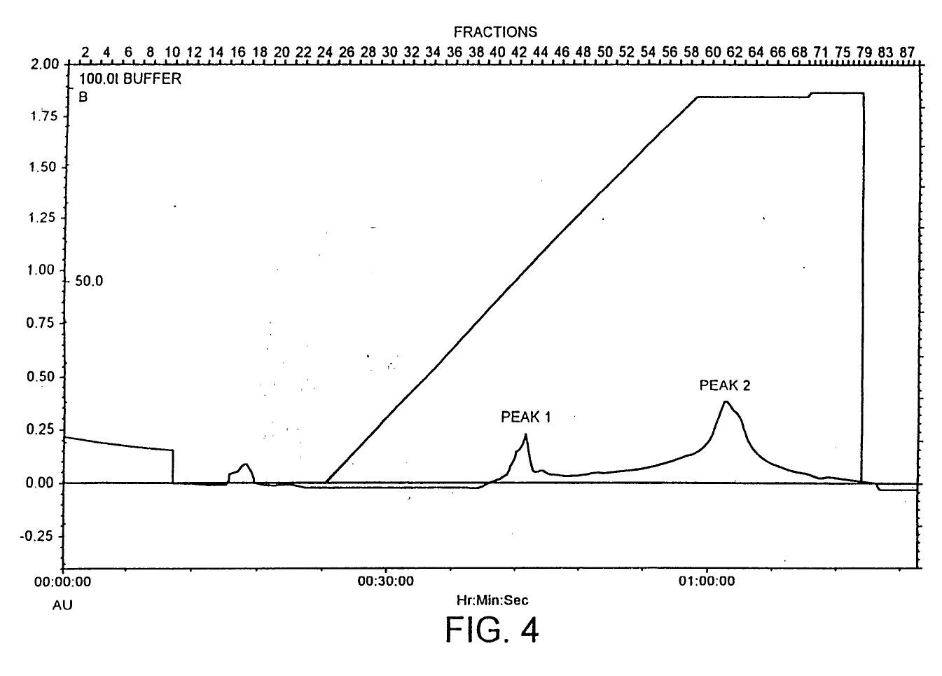

FIG. 4: is a profile from anion exchange chromatography.

FIG. 5: shows purified S. pneumoniae proteins of molecular masses 14, 16, 34 and 57 kDa

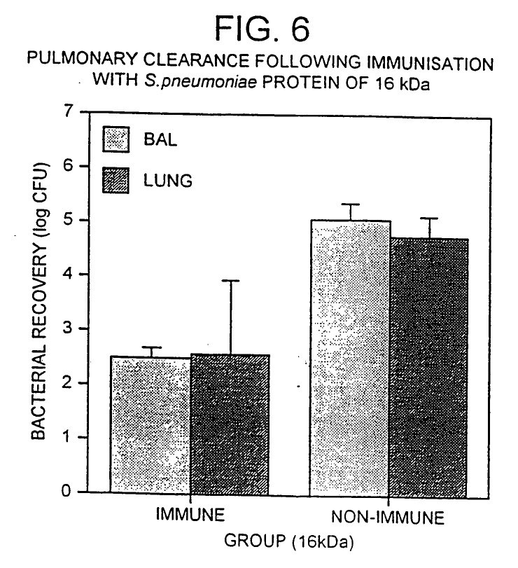

FIG. 6: is a histogram showing pulmonary clearance following immunisation with S. pneumoniae protein of 16 kDa.

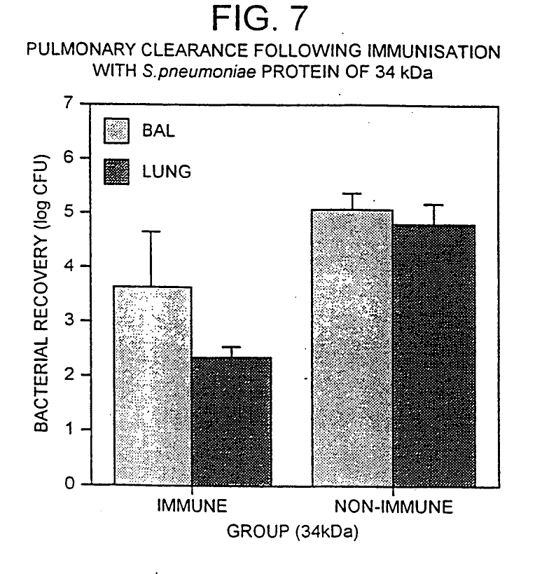

FIG. 7: is a histogram showing pulmonary clearance following immunisation with S. pneumoniae protein of 34 kDa.

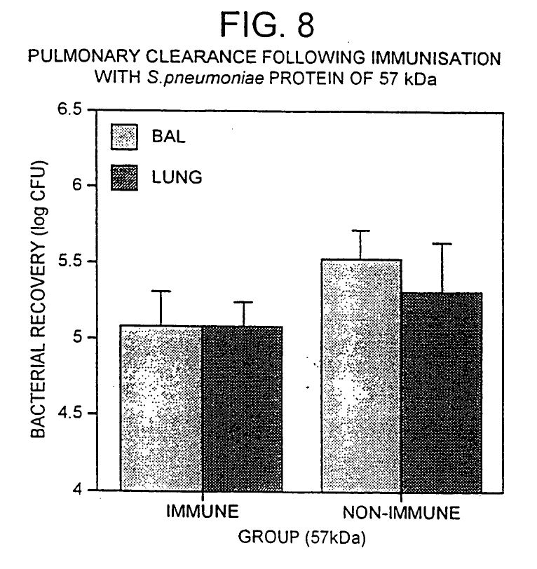

FIG. 8: is a histogram showing pulmonary clearance following immunisation with S. pneumoniae protein of 57 kDa.

### EXAMPLE 1

Isolation of Antigenic or Immunogenic Proteins from S.pneumoniae

The proteins identified herein were isolated from the cell envelope of S.pneumoniae strain NCTC 7466 (serotype 2). The strain was grown overnight to stationary phase in Bacto Tryptic Soy Broth containing 10% horse blood and 0.5% glucose at 37° C. without shaking. 10 ml of the overnight-culture was then used to inoculate 500 ml of Bacto tryptic Soy Broth containing 0.5% glucose but no blood and incubated overnight at 37° C. without shaking. The intact cells were then recovered by centrifugation at 3000 rpm (1100 g) for 25 min and resuspended in 40 ml of 50 mM Tris Maleate ph 6.8 to which protease inhibitors were added. The bacteria were disrupted in a Constant Systems cell breaker (model No. 22/40/AA/AA) using a pressure setting of 40 Kpsi. The cell homogenate was then centrifiged at 2600 rpm (1100 g) for 10 min at 4° to remove intact cells. The supernatant was then centrifuged at 15,000 rpm (27000 g) for 15 min at 4° C. to pellet the bacterial cell walls. The cell pellets were then washed twice by centrifugation in 10 ml of 50 mM Tris Maleate pH 6.8 containing protease inhibitors. Finally the cell pellets were mixed with the same buffer containing different compounds to determine which proteins would be released from the cell wall material. Proteins extracted from the cell wall material were present in the supernatant after centrifugation. Proteins extracted from the cell wall material were analysed by SDS PAGE. The photograph in FIG. 1 shows a 12% SDS PAGE gel of proteins extracted from cell wall material treated with 1 M solutions of ammonium acetate, ammonium chloride, tri-methyl ammonium chloride or Tris-HCl (pH 6.8).

The extracted proteins were concentrated using a centricon 10 spin filter and separated by SDS PAGE using various different concentrations of acrylamide. The separated proteins were then transferred to nitrocellulose membranes for isolation and N-terminal sequencing.

N-terminal sequencing was carried out according to the Applied Biosystems protocols. However, in addition, the skilled person can also carry out such sequencing according to the methods described in Matsudaira, J. Biol. Chem., 262:10035-10038 (1997).

### EXAMPLE 2

Animal Studies

A comparative trial was conducted looking at the ability of the protein mixture prepared above to protect mice against challenge with Pneumococcus. Different adjuvants were also included in the study. Antibody levels and survival to intra-nasal challenge were assessed.

Vaccination Regime

Seven week old female CBA/Ca mice were vaccinated at week 1, boosted at week 5 in the case of Freund's and Titremax adjuvants, and at week, 4 in the case of Ribi, and challenged intra-nasally with pneumococcus at week 8. A dose of 20 μg was administered subcutaneously at each vaccination. Freund's complete adjuvant+protein mixture and Titremax+protein mixture were administered s.c. in the scruff, and Ribi+protein mixture was administered s.c. on the belly.

Bleeds

Bleeds were taken at weeks 2, 4 and 6 for comparison of antibody titres.

Pneumococcal Challenge

A standard inoculum of type 4 Streptococcus pneumoniae was prepared and frozen down by passaging a culture of pneumococcus 1× through mice, harvesting from the blood of infected animals, and grown up to a predetermined viable count of around 10⁹ cfu/ml in broth before freezing down. The preparation is set out below as per the flow-chart

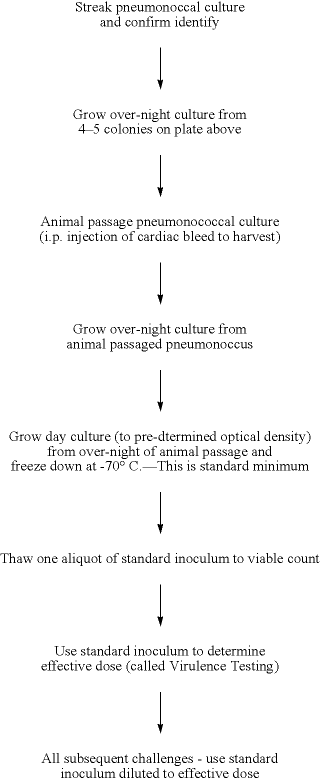

An aliquot of standard inoculum was diluted 500× in PBS and used to inoculate the mice.

Mice were lightly anaesthetised using halothane and then a 50 μl dose of 1.4×10⁵ cfu of pneumococcus was applied to the nose of each mouse. The uptake was facilitated by the normal breathing of the mouse, which was left to recover on its back.

The symptoms of the mice were recorded at set intervals during the infection.

Results

Survival Data

By 24 hrs control, unvaccinated mice were showing signs of infection and their median survival time was 49.2 hrs. Median survival times of Freund's+proteins=124.5 hrs, Ribi+proteins and Titremax+proteins=168 hrs. 2 of 6 mice in Freund's group survived, 4 of 6 mice in Ribi group survived and 6 of 6 mice survived, in Titremax group.

For mice in Freund's+proteins and Ribi+proteins that did become ill, the onset of disease was delayed in comparison to the unvaccinated control mice.

Antibody Titres

immune responses were assessed between adjuvant groups using ELISA. For Freund's, median titres were 199024 and 722119 at the 2nd and 3rd bleeds. For Ribi, median titres were 16674 and 1474354 at the 2nd and 3rd bleeds. For Titremax, median titres were 138455 and 705486 at the 2nd and 3rd bleeds.

### EXAMPLE 2

### Isolation and Purification of Antigens Bacteria

Serogroup 3 Streptococcus pneumoniae (ATCC 49619) was used to obtain antigens investigated in this study and used in homologous bacterial challenge in the animal studies. Bacterial strains were grown overnight on blood agar at 37° C. and 5% CO₂ or cultured in tryptic soya broth (Oxoid Ltd, Basingstoke, Hampshire, UK) overnight in a shaker incubator at 37° C.

Protein Purification

Extraction of Cell Wall Proteins

Aseptically, a loop full of S. pneumoniae was inoculated into 10 mL of sterile tryptone soya broth and cultured overnight in a 37° C. shaker incubator. 2×5 mL aliquots were subcultured into 2×500 mL volumes of sterile tryptone soya broth and cultured overnight in a 37° C. shaker incubator. Aseptically, a loop of bacterial suspension was removed from each culture, streaked onto blood agar and incubated overnight at 37° C. in CO₂ as a growth and contaminant check.

The bacterial culture was centrifuged at 18000×g for 20 minutes at 4° C. using a Beckman J-2™ centrifuge. The pellet was washed twice in phosphate buffered saline (PBS) by centrifugation, then resuspended in 10 mL PBS and 200 μl 10% (w/v) sodium deoxycholate and stirred at room temperature for 1 hour. The suspension was centrifuged at 27000×g for 15 minutes at 4° C., the supernatant was recovered and stirred while gradually adding ammonium sulphate to a final concentration of 70% (w/v). The suspension was centrifuged at 27000×g for 15 minutes at 4° C., the pellet redissolved in 10 mL 10 mM sodium phosphate, pH 7.0. The resuspended pellet was dialysed against 3×1 L changes of 10 mM sodium phosphate, pH 7.0 at 4° C., leaving a minimum of 2 hours between changes. The dialysed protein suspension was centrifuged for 20 minutes at 15000 rpm at 4° C., the supernatant was kept and a protein assay performed. The protein suspension was concentrated by lyophilisation and a sodium dodecyl sulphate-polyacrylamide gel electrophoresis (SDS-PAGE) analysis performed.

SDS-PAGE

The Protean II xi cell™ (Bio-Rad) was used to separate proteins according to their molecular weights. A discontinuous gel consisting of 12% (w/v) acrylamide/BIS separating gel and a 4% (w/v) acrylamide/BIS stacking (upper) gel was prepared from a 30% (w/v) stock solution of acrylamide/BIS (N,N′-methylenebisacrylamide) in Tris buffer. The polyacrylamide gel was polymerised using ammonium persulfate and TEMED. The lyophilised protein extract was suspended 1:1 (v/v) in sample buffer (0.5 M Tris HCl pH 6.8; 10% v/v) glycerol; 10% (w/v) SDS; 0.05% (w/v) bromophenol blue; 0.05% (v/v) β-mercaptoethanol), boiled for 5 minutes and then approximately 1 mL of this was loaded onto the top of the gel. Electrophoresis was performed at a constant current of 16 mA per gel until the dye front passed through the stacker and then increased to 24 mA for electrophoresis through the resolving gel. The average running time was between 4 and 5 hours. The separated proteins were then recovered by electroelution using the BIORAD™ flat bed electroeluter for 1 hour at 200V and a maximum of 0.2 mA into 30 individual tubes. Protein composition of the recovered fractions was assessed by analytical SDS-PAGE and either Coomassie or Silver staining of proteins. Analytical SDS-PAGE was performed using a Mini-protean II™ cell (Bio-Rad) at a constant 200V for about 45 minutes. Protein concentrations were determined using the Pierce Micro BCA™ protein assay and comparison with albumin standards.

SDS Removal from Purified Proteins

Samples containing SDS were treated with a 200 μL volume of 100 mM potassium phosphate per 1 mL of sample and left on ice for 60 minutes. The sample was centrifuged at 10000×g for 20 minutes at 4° C. in a microcentrifuge. The supernatant was recovered and desalted by overnight dialysis against nanopure water.

Liquid Chromatography Separation

Anion Exchange Liquid Chromatography

The extracted proteins were additionally purified by anion exchange chromatography and separated according to their molecular charge interactions. The column (Q5 column Bio-Rad) was equilibrated with a low salt buffer (20 mM Tris-HCl, pH 8.45) at a flow rate of 1 mL/min for 10 minutes. Lyophilised cell wall extracts were resuspended in the same buffer to a concentration of 5mg per mL and loaded onto the column. Proteins were eluted using an increasing salt gradient by gradually increasing the proportion of 20 mM Tris-HCl, 500 mM sodium chloride, pH 8.6 passed through the column. Fractions were recovered, lyophilised and assessed by analytical SDS-PAGE. Fractions from multiple runs were pooled and proteins were further purified by preparative SDS-PAGE and electroelution as previously described.

Results

The methods described above successfully purified ten proteins of different molecular weights which were able to be assessed in animal immunisation studies as described in Example 4 below. The most active proteins purified had molecular masses of 12-14 kDa, 16 kDa, 34 kDa and 57 kDa. In total, 23 different proteins were separated in yields ranging from 20 to 500 μg in a 6 L culture with a total protein concentration of cell wall extract of from 25-30 mg. FIG. 2 shows the profile of the cell wall extract and the different proteins separated by electroelution from the crude protein extract Not all proteins eluted from the gel as a single protein band; some fractions were composed of 2 or 3 different proteins.

Elution Profile for Cell Wall Proteins Using Anion Exchange Chromatography

The elution profile from anion exchange chromatography is shown in FIG. 3. The first peak represents elution of unbound proteins. The subsequent two major peaks contained most of the proteins that were eluted with increasing salt concentration. The proteins in these peaks were further purified by SDS-PAGE.

### EXAMPLE 3

### N-terminal Sequence Analysis

The N-terminal sequence of the proteins was determined from an excised band from an analytical SDS-PAGE. Analyses were performed by the Biomolecular Resource Unit, The John Curtin School of Medical Science (Australian Capital Territory, Australia).

TABLE 1
Amino Acid Sequence Analysis Results of the Purified Proteins

| Protein | | |
|---|---|---|
| Molecular | | |
| Mass (kDa) | N-terminal Sequence | Homology Identity |
| 12-14 | ALNIENIIAEIKEAS | S. pneumoniae ribosomal protein |
| 16 | To be confirmed | |
| 34 | AKYEILYIIRPNIEE | S. pneumoniae ribosomal protein |
| 57 | RIIKFVYAK | REV protein/fragment |

To assist in the characterisation of the proteins, the information obtained from the partial amino acid sequence was searched through the GenBank databases to determine homology to known protein sequences. It was found that the 12-14 kDa protein has a 100% sequence homology match with that of a 12 kDa protein from S. pneumoniae. The 34 kDa protein was determined to have a 78% sequence homology with that of a protein from Bacillus subtillus. Limited investigation on both proteins has postulated that they are ribosomal proteins, yet this remains to be confirmed.

According to a study by Koberg et al, (Microbiology, 143(1), 55-61 (January 1997)), two monoclonal antibodies against Streptococcus pneumoniae reacted with a highly conserved epitope on eubacterial L7/L12 ribosomal proteins. A high degree of amino acid sequence homology was found across 66 eubacteria, representing 27 different species. Our approximate 12-14 kDa protein had a 100% sequence match with the 12 kDa protein from this study (Kolberg et al). Since this protein is postulated to be toxic and is conserved across species, even gram negative bacteria, it is of great interest to proceed with further studies to characterise the protein and determine its involvement in the virulence of disease associated with this organism.

The 34 kDa protein appears to be a novel protein of S. pneumoniae, since the closest match was a 78% match with Bacillus subtillus ribosomal protein S6. Ribosomal protein S6 has a role in initiation of chromosome replication in the cell cycle (Moriya et al, Nucleic Acids Res., 13, 2251-2265 (1985)). The homology match reveals a degree of conservation of this protein across species.

### EXAMPLE 4

### Mouse Lung Clearance Model Animals

Balb/c mice, 6-10 weeks old were housed and maintained in a pathogen free environment with free access to sterilised food and water.

Preparation of Live Bacteria

Bacteria were grown overnight on blood agar plates at 37° C. and 5% CO₂. The bacteria were harvested and washed twice in sterile PBS by centrifugation at 10000×g at room temperature. The bacterial concentration was determined by optical density at 405 nm and calculated from a regression curve. the accuracy of the concentration for viable bacterial count was confirmed by titration and overnight culture.

Immunisation Regime

Mice were initially immunised on day 0 by Peyer's patches inoculation and boosted by intratracheal administration 14 days later. On day 21, these mice were challenged with live S. pneumoniae.

Pever's Patch Immunisation

The mice were sedated by a subcutaneous injection of 0.25 mL ketamine/xylazine at a dosage of 5 mg/ml ketamine hydrochloride; 2mg/ml xylazine hydrochloride. The small intestine was exposed through a mid-line abdominal incision and the protein injected subserosal into each Peyer's patch. The immunisation protein was prepared by emulsifying 2.5 μg/μL protein in a 1:1 ratio with incomplete Freund's adjuvant (Sigma Immunochemicals, St Louis, Mich., USA) and a total concentration of 10 μg protein administered to each animal.

Intratracheal Inoculation of Mice

On day 14, mice received an intratracheal boost. The mice were sedated by intravenous injection with 20 mg saffan per kg of body weight. 10 μg protein in PBS in a total volume of 20 μL was delivered via the trachea into the lungs with a 22.1/2G catheter.

Pulmonary Challenge

On day 21, the mice received a live bacterial challenge. The mice were sedated with saffan as described above, and an inoculum of 1×10⁷ CFU in 20 μL of live S. pneumoniae was introduced into the lungs via the trachea as for the intratracheal boost. Five hours following the challenge, the mice were euthanased by an intraperitoneal injection of 0.2 mL of sodium pentobarbital.

Blood was collected by heart puncture and the separated serum stored below −20° C. prior to analysis. The trachea was exposed and the lungs were lavaged by insertion and removal of 0.5 mL sterile PBS. The recovered fluid (BAL) was assessed for bacterial recovery by plating 10 fold serial dilutions onto blood agar for CFU determination. An aliquot was removed for cytospin slide preparation, staining and differential cell counts. The BAL was then centrifuged for 10 min at 1000 rpm at 4° C. and the supernatant stored below −20° C. until required. The pellet was resuspended in PBS and methylene blue and the total number of white cells in the BAL were counted. The lungs were removed following lavage, placed in 2 mL sterile PBS and homogenised. The lung homogenate was assessed by plating 10-fold serial dilutions onto blood agar for CFU determination. Results are presented only for the proteins which showed significant degrees of pulmonary clearance from the lungs.

Results

Three proteins assessed in immunisation and bacterial challenge showed significant degrees of pulmonary clearance from the lungs. These were proteins with molecular masses of 16, 34 and 57 kDa and identified in Table 1 above. The results of the bacterial clearance and comparison with the recovery in non-immune mice challenged at the same time are shown in Table 2 below and graphically represented in FIGS. 5 to 7. A fourth protein of significance was the 12-14 kDa protein. In three separate immunisation studies and using freshly isolated protein in each case, immunisation was lethal to the mice with most animals not recovering from anaesthesia. Thirteen out of seventeen mice died over three experiments leaving only a maximum of two surviving in any given experiment. This protein is of interest as a toxin and potential virulence component of S. pneumoniae. Identification of the toxic component and detoxification of the protein may result in a highly efficacious antigen. This protein has been previously identified through monoclonal antibody assay (see above) as being present in a large number of bacteria. However, there is no evidence in the literature that it has been tested as a vaccine antigen.

TABLE 2
Pulmonary Clearance Following Immunisation With Purified Proteins

| | BAL | LUNG | Total White Cell |
|---|---|---|---|
| | (log₁₀ CFU) | (log₁₀ CFU) | Count in BAL (×10⁶) |
| 16 kDa | | | |
| Immune | 2.49 ± 0.16 | 2.56 ± 1.32 | 1.70 ± 1.01 |
| Non-immune | 5.07 ± 0.26 | 4.77 ± 0.36 | 1.65 ± 0.41 |
| 34 kDa | | | |
| Immune | 3.66 ± 0.99 | 2.38 ± 0.15 | 0.81 ± 0.17 |
| Non-immune | 5.07 ± 0.26 | 4.77 ± 0.36 | 1.65 ± 0.41 |
| 57 kDa | | | |
| Immune | 5.1 ± 0.20 | 5.1 ± 0.13 | 0.087 ± 0.047 |
| Non-immune | 5.5 ± 0.20 | 5.3 ± 0.31 | 0.032 ± 0.015 |

1. A protein or polypeptide obtainable from S. pneumoniae selected from:
(i) one having a molecular weight of 55 kDa, as determined by SDS/PAGE, and having the N-terminal sequence VEPKAKPADPSVV (SEQ ID NO:1);
(ii) one having a molecular weight of 50 kDa, as determined by SDS/PAGE, and having the N-terminal sequence NDRLVATQSADGRNESVLMSIET (SEQ ID NO:2);
(iii) one having a molecular weight of 85 kDa, as determined by SDS/PAGE, and having the N-terminal sequence EDTTNSRFGSQFDKYRQPNAEPDHSHDAVSADNSTAHNRFGYGFAIGSKYIRYD (SEQ ID NO:3);
(iv) one having a molecular weight of 38 kDa, as determined by SDS/PAGE, and having the N-terminal sequence DKYRQPNAEPDDHHYAV (SEQ ID NO:4);
(v) one having a molecular weight of 30 kDa, as determined by SDS/PAGE, and having the N-terminal sequence DAVSAD (SEQ ID NO:5) or SETNVY (SEQ ID NO:6);
(vi) one having a molecular weight of 32 kDa, as determined by SDS/PAGE, and having the N-terminal sequence DKVDGLSAKPDILKP (SEQ ID NO:7);
(vii) one having a molecular weight of 43 kDa, as determined by SDS/PAGE, and having the N-terminal sequence ELKEEG(W)VVK (SEQ ID NO:8);
(viii) one having a molecular weight of 100 kDa, as determined by SDS/PAGE, and having the N-terminal sequence EVHA 1 (SEQ ID NO:9);
(ix) one having a molecular weight of <14 kDa, as determined by SDS/PAGE, and having the N-terminal sequence MKLNEVKEFVKELRAET (SEQ ID NO:10);
(x) one having a molecular weight of <14 kDa, as determined by SDS/PAGE, and having the N-terminal sequence AKYEILYIERPNIEEFAK (SEQ ID NO:11);
(xi) one having a molecular weight of <14 kDa, as determined by SDS/PAGE, and having the N-terminal sequence I(R)LTRM(E)GGKKKP(K)FYY (SEQ ID NO:12);
(xii) one having a molecular weight of 16 kDa, as determined by SDS/PAGE, and having the N-terminal sequence VMTDPIADXLXRI (SEQ ID NO:13);
(xiii) one having a molecular weight of 27.5 kDa, as determined by SDS/PAGE, and having the N-terminal sequence (VA)(KE)LVFARHGE(LT)E(NK) (SEQ ID NOS:14-29);
(xiv) one having a molecular weight of 44 kDa, as determined by SDS/PAGE, and having the N-terminal sequence IITDVYAREVLDSRGNPTL (SEQ ID NO:30).
(xv) one having a molecular weight of 12-14 kDa as determined by SDS PAGE under reducing conditions and has the following amino terminal sequence:

| | |
|---|---|
| A L N I E N I I A E I K I A S | |
| Ala Leu Asn Ile Glu Asn Ile Ile Ala Glu Ile Lys Ile Ala Ser | (SEQ ID NO:31) |

(xvi) is a reduced toxicity variant or fragment of the protein defined in (xv) above;
(xvii) one having a molecular weight of about 16 kDa. as determined by SDS PAGE under reducing conditions; or
(xviii) has a molecular weight of about 57 kDa as determined by SDS PAGE under reducing conditions and has the following amino terminal sequence:

| | |
|---|---|
| R I I K F V Y A K | |
| Arg Ile Ile Lys Phe Val Tyr Ala Lys. | (SEQ ID NO:32) |

2. A protein or polypeptide as claimed in claim 1 which is in substantially pure form.

3. A homologue, derivative or fragment-of a protein or polypeptide as claimed in claim 1.

4. One or more antigenic fragments of a protein or polypeptide as claimed in claim 1.

5. A nucleic acid molecule comprising or consisting of a sequence which is:
(i) a DNA sequence coding for a protein or polypeptide as defined in claim 1 or its homologue, derivative, or fragment thereof or its RNA equivalent.
(ii) a sequence which is complementary to any of the sequences of (i); and
(iii) a sequence which has substantial identity with any of those of (i) and (ii).

6. A vector comprising a nucleic acid molecule as defined in claim 5.

7. A host cell comprising a vector as defined in claim 6.

8. The use of a protein or polypeptide as claimed in claim 1 or of its homologue, derivative, or fragment thereof in medicine.

9. An immunogenic/antigenic composition comprising one or more proteins or polypeptides as defined in claim 1 or homologues or derivatives thereof and/or fragments of any of these.

10. A composition as claimed in claim 9 which composition is a vaccine or is for use in a diagnostic assay.

11. A vaccine composition comprising one or more nucleic acid molecules as defined in claim 5.

12. An antibody raised against and/or capable of binding to a protein or polypeptide as defined in claim 1 or its homologue, derivative or fragment thereof.

13. A method for the detection/diagnosis of S. pneumoniae which comprises the step of bringing into contact a sample to be tested with a protein or polypeptide as defined in claim 1 or its homologue, derivative or fragment thereof.

14. A method for the detection/diagnosis of S. pneumoniae which comprises the step of bringing into contact a sample to be tested and one or more antibodies capable of binding a protein or polypeptide as defined in claim 1 its homologue, derivative or a fragment thereof.

15. A method for the detection/diagnosis of S. pneumoniae which comprises the step of bringing into contact a sample to be tested with at least one nucleic acid molecule as defined in claim 5.

16. A method of vaccinating a subject against S. pneumoniae which comprises the step of administering to a subject a protein or polypeptide as defined in claim 1 or its derivative, homologue or fragment thereof.

17. A method of vaccinating a subject against S. pneumoniae which comprises the step of administering to a subject a nucleic acid molecule as defined in claim 5.

18. A method for the prophylaxis or treatment of S. pneumoniae infection which comprises the step of administering to a subject a protein or polypeptide as defined in claim 1 or its derivative, homologue or fragment thereof.

19. A method for the prophylaxis or treatment of S. pneumoniae infection which comprises the step of administering to a subject a nucleic acid molecule as defined in claim 5.

20. A kit for use in detecting/diagnosing S. pneumoniae infection comprising one or more proteins or polypeptides as defined in claim 1 or its homologue, derivative or fragment thereof.

21. A kit for use in detecting/diagnosing S. pneumoniae infection comprising one or more nucleic acid molecules as defined in claim 5.

22. A method of determining whether a protein or polypeptide as defined in claim 1 or its homologue, derivative, or fragment thereof represents a potential anti-microbial target which comprises inactivating said protein or polypeptide and determining whether S. pneumoniae is still viable, in vitro or in vivo.

23. The use of an agent capable of antagonising, inhibiting or otherwise interfering with the function or expression of a protein or polypeptide as defined in claim 1 or its homologue, derivative, or fragment thereof in the manufacture of a medicament for use in the treatment or prophylaxis of S. pneumoniae infection.

24. A process for the preparation of an isolated and purified protein the process comprising the following steps:
(a) preparing cultures of S. pneumoniae, growing the cultures under appropriate conditions and harvesting them, followed by washing with centrifugation to yield a washed cell pellet;
(b) resuspending the washed cells in an appropriate buffer followed by disruption of the cells;
(c) centrifuging to remove cell debris and obtaining the supernatant containing soluble cell proteins;
(d) subjecting the solution obtained to anion exchange chromatography with a sodium chloride gradient elution, and pooling the fractions corresponding to each separate peak;
(e) suspending the protein fractions in a buffer comprising 0.5 M Tris HCl pH 6.8; 10%)(v/v) glycerol; 10% (w/v) SDS; 0.05% (w/v) bromophenol blue and 0.05% (v/v) β-mercaptoethanol; boiling the mixture and then purifying by SDS-PAGE using a 12% (w/v) acrylamide/BIS separating gel with a 4% (w/v) acrylamide/BIS stacking gel, run at 16 mA in the stacking gel and 24 mA in the resolving gel;
(f) selecting a fraction containing a protein having a molecular weight of 12-14 kDa, 16 kDa, 34 kDa, or 57 kDa and isolating the protein from the selected fraction.


SEQUENCE LISTING (34 sequences)

SEQ ID NO: 1 - protein, 13 aa, Streptococcus pneumoniae
VEPKAKPADPSVV

SEQ ID NO: 2 - protein, 23 aa, Streptococcus pneumoniae
NDRLVATQSADGRNESVLMSIET

SEQ ID NO: 3 - protein, 54 aa, Streptococcus pneumoniae
EDTTNSRFGSQFDKYRQPNAEPDHSHDAVSADNSTAHNRFGYGFAIGSKYIRYD

SEQ ID NO: 4 - protein, 17 aa, Streptococcus pneumoniae
DKYRQPNAEPDDHHYAV

SEQ ID NO: 5 - protein, 6 aa, Streptococcus pneumoniae
DAVSAD

SEQ ID NO: 6 - protein, 6 aa, Streptococcus pneumoniae
SETNVY

SEQ ID NO: 7 - protein, 15 aa, Streptococcus pneumoniae
DKVDGLSAKPDILKP

SEQ ID NO: 8 - protein, 10 aa, Streptococcus pneumoniae
ELKEEGWVVK

SEQ ID NO: 9 - protein, 4 aa, Streptococcus pneumoniae
EVHA

SEQ ID NO: 10 - protein, 17 aa, Streptococcus pneumoniae
MKLNEVKEFVKELRAET

SEQ ID NO: 11 - protein, 18 aa, Streptococcus pneumoniae
AKYEILYIERPNIEEFAK

SEQ ID NO: 12 - protein, 17 aa, Streptococcus pneumoniae
IRLTRMEGGKKKPKFYY

SEQ ID NO: 13 - protein, 13 aa, Streptococcus pneumoniae
  SITE (9): Xaa= any amino acid
VMTDPIADXLXRI

SEQ ID NO: 14 - protein, 13 aa, Streptococcus pneumoniae
VKLVFARHGELEN

SEQ ID NO: 15 - protein, 13 aa, Streptococcus pneumoniae
VKLVFARHGELEK

SEQ ID NO: 16 - protein, 13 aa, Streptococcus pneumoniae
VKLVFARHGETEN

SEQ ID NO: 17 - protein, 13 aa, Streptococcus pneumoniae
VKLVFARHGETEK

SEQ ID NO: 18 - protein, 13 aa, Streptococcus pneumoniae
VELVFARHGELEN

SEQ ID NO: 19 - protein, 13 aa, Streptococcus pneumoniae
VELVFARHGELEK

SEQ ID NO: 20 - protein, 13 aa, Streptococcus pneumoniae
VELVFARHGETEN

SEQ ID NO: 21 - protein, 13 aa, Streptococcus pneumoniae
VELVFARHGETEK

SEQ ID NO: 22 - protein, 13 aa, Streptococcus pneumoniae
AKLVFARHGELEN

SEQ ID NO: 23 - protein, 13 aa, Streptococcus pneumoniae
AKLVFARHGELEK

SEQ ID NO: 24 - protein, 13 aa, Streptococcus pneumoniae
AKLVFARHGETEN

SEQ ID NO: 25 - protein, 13 aa, Streptococcus pneumoniae
AKLVFARHGETEK

SEQ ID NO: 26 - protein, 13 aa, Streptococcus pneumoniae
AELVFARHGELEN

SEQ ID NO: 27 - protein, 13 aa, Streptococcus pneumoniae
AELVFARHGELEK

SEQ ID NO: 28 - protein, 13 aa, Streptococcus pneumoniae
AELVFARHGETEN

SEQ ID NO: 29 - protein, 13 aa, Streptococcus pneumoniae
AELVFARHGETEK

SEQ ID NO: 30 - protein, 19 aa, Streptococcus pneumoniae
IITDVYAREVLDSRGNPTL

SEQ ID NO: 31 - protein, 15 aa, Streptococcus pneumoniae
ALNIENIIAEIKIAS

SEQ ID NO: 32 - protein, 9 aa, Streptococcus pneumoniae
RIIKFVYAK

SEQ ID NO: 33 - protein, 15 aa, Streptococcus pneumoniae
ALNIENIIAEIKEAS

SEQ ID NO: 34 - protein, 15 aa, Streptococcus pneumoniae
AKYEILYIIRPNIEE